(19) United States
(12) Patent Application Publication
(10) Pub. No.: US 2003/0215785 A1
(43) Pub. Date: Nov. 20, 2003

(54) Solution containing platelet activation inhibitors for pathogen reducing and storing blood platelets

(75) Inventors: Laura Goodrich, Lakewood, CO (US); Raymond P. Goodrich, Lakewood, CO (US); Deanna Gampp, Thornton, CO (US); Robert Owen Lockerbie, Littleton, CO (US)

Correspondence Address: GAMBRO, INC, PATENT DEPARTMENT, 10810 W COLLINS AVE, LAKEWOOD, CO, 80215, US

(73) Assignee: Gambro, Inc., Lakewood, CO

(21) Appl. No.: 10/425,282

(22) Filed: Apr. 28, 2003

Related U.S. Application Data
(60) Provisional application No. 60/375,670, filed on Apr. 26, 2002.

(51) Int. Cl. A01N 1/02

(52) U.S. Cl. 435/2

(57) ABSTRACT

This invention relates to the addition of platelet activation inhibitors to solutions used in the pathogen reduction and subsequent storage of platelets. More particularly, the invention relates to the addition of adenylate cyclase stimulators and phosphodiesterase inhibitors to a platelet pathogen reduction and storage solution.

### CROSS REFERENCE To RELATED APPLICATIONS

This application claims priority to U.S. Provisional patent application No. 60/375,670 filed Apr. 26, 2002.

### BACKGROUND

Contamination of blood supplies with infectious microorganisms such as HIV, hepatitis and other viruses and bacteria presents a serious health hazard for those who must receive transfusions of whole blood or administration of various blood components such as platelets, red cells, blood plasma, Factor VIII, plasminogen, fibronectin, anti-thrombin III, cryoprecipitate, human plasma protein fraction, albumin, immune serum globulin, prothrombin, plasma growth hormones, and other components isolated from blood plasma. Blood screening procedures which are currently available may miss contaminants. Thus, there is a need for sterilization procedures that effectively neutralize all infectious viruses and other microorganisms but do not damage cellular blood components, do not degrade desired biological activities of proteins, and preferably do not need to be removed prior to administration of the blood product to the patient.

One method used to sterilize blood and blood components requires the use of photosensitizers, compounds which absorb light of a defined wavelength and transfer the absorbed energy to an energy acceptor.

It is known in the art to use photosensitizers as a component of solutions used to photodecontaminate contaminants which may be in blood and blood products. U.S. Pat. No. 5,709,991 to Lin et al. teaches the use of the photosensitizer psoralen for photodecontamination of platelet preparations and removal of photolysed psoralen afterward. U.S. Pat. No. 5,459,030 also issued to Lin teaches a platelet storage solution containing 8-methoxypsoralen for use in a pathogen reduction process of platelets. U.S. Pat. Nos. 5,712,085, 5,908,742, 5,955,256, 5,965,349, 6,017,691 and 6,251,580 all disclose solutions for use in the pathogen reduction of blood which include psoralen or psoralen derivatives as the photosensitizer.

It has been found that platelets which have been treated with a photosensitizer and light to reduce pathogens which may be present are likely to become activated during long term storage after such a treatment. In addition to causing aggregation of the platelets, the pathogen reduction process also may yield high GMP-140 expression and low ESC (extended shape change) response by day 5 of storage, both of which may be indications of cytoskeletal changes in the platelets. Such changes may be indications of platelet damage which may have occurred as a result of the storage conditions. It is therefore important to improve the quality of stored pathogen reduced platelets. It is against this background that the present invention is directed.

### SUMMARY OF THE INVENTION

The invention generally relates to synthetic media for use in the pathogen reduction and subsequent storage of platelets. By the term “synthetic media” the present invention intends to indicate aqueous solutions (e.g., phosphate buffered, aqueous salt solutions) other than those naturally occurring (e.g., plasma, serum, etc.). However, it is contemplated that such synthetic media may also be used in combination with naturally occurring fluids. The platelet pathogen reduction and storage solution of this invention comprises a fluid containing platelets, a photosensitizer, and at least one platelet activation inhibitor.

### BRIEF DESCRIPTION OF THE DRAWINGS

FIGS. 1-6 are standard measures used to determine the structural integrity, respiratory activity and functionality of stored platelets. These standard measures may also be used to measure the cell quality of platelets which have been pathogen reduced and stored over five days in solutions with and without platelets activation inhibitor additives.

FIG. 1 is a graph of the cell count of pathogen reduced platelets over 5 days of storage.

FIG. 2 is a graph showing the changes in pH of pathogen reduced platelets over 5 days of storage.

FIG. 3 shows glucose consumption of pathogen reduced platelets over 5 days of storage.

FIG. 4 shows lactose production of pathogen reduced platelets over a 5 day storage period.

FIG. 5 is a measure of the % recovery from HSR of pathogen reduced platelets over 5 days of storage.

FIG. 6 shows expression of P-selectin by platelets stored over 5 days.

FIG. 7 shows VEGF release by pathogen reduced platelets over five days of storage.

FIG. 8 shows RANTES release by pathogen reduced platelets over five days of storage.

FIG. 9 is a graph of the upregulation of cAMP in pathogen reduced platelets over five days of storage.

FIG. 10 shows an embodiment of this invention using a bag to contain the fluid containing platelets being treated with the photosensitizer and platelet activation inhibitor additives, and a shaker table to agitate the fluid while exposing to photoradiation from a light source.

### DETAILED DESCRIPTION

This invention provides a synthetic aqueous solution for prolonging the preservation of human blood platelets either during or after a pathogen reduction procedure. The method uses inhibitors of platelet activation which help platelets retain their functional integrity during prolonged storage after a pathogen reduction procedure, or during the pathogen reduction procedure itself. This is accomplished by inhibiting normal platelet function, so as to help keep platelets from becoming activated during the pathogen reduction process or afterwards during storage.

Platelet activation during a pathogen reduction procedure and subsequent storage is undesirable. However, treated platelets must retain the ability to function normally and become activated in response to a stimulus when they are taken out of long term storage and transfused into a patient.

There are three parameters of platelet activity that are commonly used to measure whether pathogen reduced platelets have retained their functional ability after storage. These tests can also be used to measure platelet function after a pathogen reduction procedure. Platelet number, hypotonic stress response, and agonist-induced platelet aggregation are some commonly used measures which may be used to measure platelet cell quality during and after a pathogen reduction procedure.

Hypotonic stress response is an assay used to determine if platelets have retained metabolic viability. This assay is a photometric measurement of the platelets' ability to overcome the addition of a hypotonic solution. This activity reflects cell function (i.e. a functional membrane water pump) and is indicative of platelet recovery following storage. Hypotonic stress response has been demonstrated to be an important indicator of platelets' ability to survive in circulation following transfusion. Consequently, hypotonic stress response represents a crucial parameter for evaluating platelet biochemistry following storage.

Potential for aggregation is another feature that demonstrates whether blood platelets have maintained their functional integrity during storage. This potential is measured using agonist-induced aggregation, where the aggregation or clumping of platelets is the response. The agonists, ADP and collagen, are used to induce aggregation to determine if platelets have retained their ability to aggregate in response to a stimulus. In addition, when performing aggregation responses one can detect the presence of spontaneous aggregation, that is the platelets adhering to each other without the addition of an agonist. The occurrence of spontaneous aggregation has been correlated with removal of platelets from the circulation and hence have short survival times in a transfusion recipient.

The platelet activation inhibitor system of this invention is based on the addition to a pathogen reduction and/or storage solution of specific second messenger effectors which help stabilize the platelets during the pathogen reduction process as well as during storage to help the pathogen reduced platelets remain viable and retain their functional activity.

Many synthetic solutions to prevent the activation of platelets during storage are known. For example, U.S. Pat. No. 5,919,614 to Livesay et al. discloses a solution for the long term storage of platelets at reduced temperatures. The storage solution of this patent uses a platelet inhibitor system to keep platelets from activating during storage. U.S. Pat. No. 4,994,367 to Bode at al. also discloses a solution for the long-term storage of platelets which includes platelet activation inhibitors. Neither of these storage solutions are directed towards a solution for pathogen reduction and subsequent storage of platelets.

The term “platelet activator inhibitor” as used herein includes adenylate cyclase stimulators and phosphodiesterase inhibitors.

The addition of prostaglandins to platelet storage solutions has been suggested as a potential resolution to the problem of platelet activation and subsequent aggregation during storage. Prostaglandins help to inhibit platelet activation through stimulation of adenylate cyclase, which produces a rapid (but transient) increase in intracellular cAMP (adenosine 3′, 5′-cyclic phosphate). In platelets, cAMP helps block activation pathways and cytoskeletal changes which occur upon platelet activation. cAMP (alone and in concert with cGMP) has been implicated in preventing the up regulation of proteins which help to trigger platelet activation, such as p-selectin and gp2bIIa.

In the present invention, the adenylate cyclase stimulator is added to the pathogen reduction and/or storage solution in an amount effective to increase the production of cAMP in the blood platelets and prevent activation of the irradiated platelets during the pathogen reduction process and storage. Exemplary adenylate cyclase stimulators which may be used in the present invention to increase intracellular cAMP and inhibit platelet activation include Prostaglandin E1 (PGE₁) and forskolin. These adenylate cyclase stimulators may be used alone, in combination with other adenylate cyclase stimulators or with other platelet activation inhibitors. PGE₁ may be used at a final concentration between about 100 nM and 500 nM, preferably about 150 nM. Forskolin may be used at a final concentration between about 1 μM and about 100 μM, preferably about 3 μM.

Also included in the pathogen reduction/storage solution of this invention are phosphodiesterase inhibitors. Phosphodiesterase inhibitors help potentiate the inhibitory effects of prostaglandins, further increasing intracellular cAMP, and preventing platelet activation during storage. Compounds which may be used in the present invention include methylxanthines such as theophylline, caffeine, xanthine, theobromine, aminophylline, oxtriphylline, dyphylline, pentoxifylline, isobutulmethylxanthine, dipyramole, and papaverine. Derivatives of methylxanthine may also be used. The above phosphodiesterase inhibitors may be used alone, in combination with other phosphodiesterase inhibitors or with other platelet inhibitors. Exemplary phosphodiesterase inhibitors which may be used in the present invention include theophylline and caffeine. Theophylline may be used at a final concentration of between about 0.90 mM and 2.2 mM, preferably about 0.95 mM. Caffeine may be used at a final concentration between about 0.90 mM and 2.2 mM, also preferably about 0.95 mM.

The amount of platelet activation inhibitor additives to be mixed with the platelets will be an amount sufficient to adequately maintain the intracellular cAMP levels in platelets so as to prevent platelets from becoming activated, but not enough to be toxic to the platelets. The platelet activation inhibitor additives may be added directly to the platelets, or may be added in a pre-mixed aqueous solution, for example water, storage buffer or a suspension solution.

The pathogen reduction componds useful in this invention include any photosensitizers known to the art to be useful for inactivating microorganisms or other infectious particles. A “photosensitizer” is defined as any compound which absorbs radiation at one or more defined wavelengths and subsequently utilizes the absorbed energy to carry out a chemical process. Examples of such photosensitizers include porphyrins, psoralens, dyes such as neutral red, methylene blue, acridine, toluidines, flavine (acriflavine hydrochloride) and phenothiazine derivatives, coumarins, quinolones, quinones, and anthroquinones. Photosensitizers of this invention may include compounds which preferentially adsorb to nucleic acids, thus focusing their photodynamic effect upon microorganisms and viruses with little or no effect upon accompanying non-nucleated cells or proteins. Other photosensitizers are also useful in this invention, such as those using singlet oxygen-dependent mechanisms.

Most preferred are endogenous photosensitizers. The term “endogenous” means naturally found in a human or mammalian body, either as a result of synthesis by the body or because of ingestion as an essential foodstuff (e.g. vitamins) or formation of metabolites and/or byproducts in vivo. Examples of such endogenous photosensitizers are alloxazines such as 7,8-dimethyl-10-ribityl isoalloxazine (riboflavin), 7,8,10-trimethylisoalloxazine (lumiflavin), 7,8-dimethylalloxazine (lumichrome), isoalloxazine-adenine dinucleotide (flavine adenine dinucleotide [FAD]), alloxazine mononucleotide (also known as flavine mononucleotide [FMN] and riboflavine-5-phosphate), vitamin Ks, vitamin L, their metabolites and precursors, and napththoquinones, naphthalenes, naphthols and their derivatives having planar molecular conformations. The term “alloxazine” includes isoalloxazines. Endogenously-based derivative photosensitizers include synthetically derived analogs and homologs of endogenous photosensitizers which may have or lack lower (1-5) alkyl or halogen substituents of the photosensitizers from which they are derived, and which preserve the function and substantial non-toxicity thereof. Such endogenously-based derivative photosensitizers which may be used in this invention are disclosed in U.S. Pat. No. 6,268,120 to Platz et al., and discloses alloxazine derivatives which may also be used to inactivate microorganisms contained in blood or blood components. This patent is incorporated by reference into the present invention to the amount not inconsistent.

When endogenous photosensitizers are used, particularly when such photosensitizers are not inherently toxic or do not yield toxic photoproducts after photoradiation, no removal or purification step is required after decontamination, and the treated product can be directly administered to a patient by any methods known in the art. Preferred endogenous photosensitizers are:

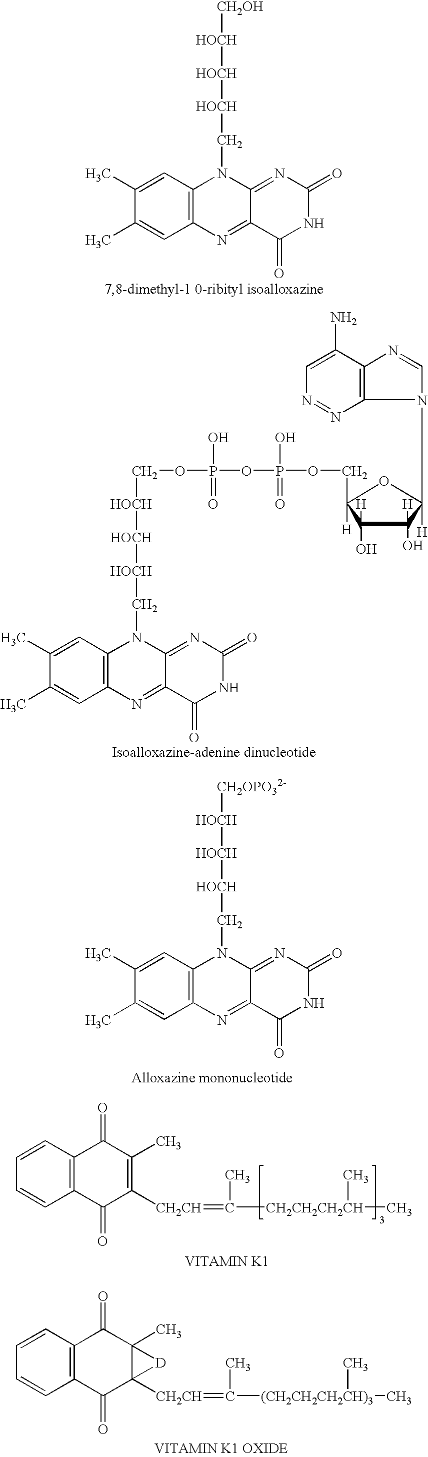

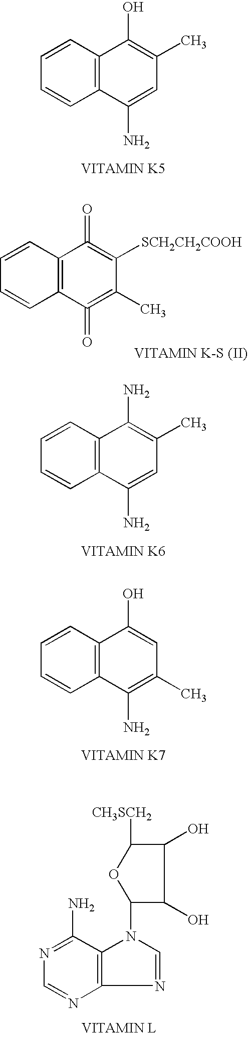

The method of this invention requires mixing the pathogen reduction compound and platelet activation inhibitors with the fluid containing platelets. Mixing may be done by simply adding the pathogen reduction compound and platelet activation inhibitors or a solution containing the pathogen reduction compound and platelet activation inhibitors directly to the fluid to be pathogen reduced. Platelet activation inhibitors may be added to the platelets separately from the pathogen reduction compound or they can be added together.

The fluid containing at least platelets and pathogen reduction compound and platelet activation inhibitors is exposed to photoradiation for a time sufficient to reduce any pathogens which may be contained in the fluid. The wavelength used will depend on the type of pathogen reduction compound selected and the type of blood component being pathogen reduced. For platelets, the light source may provide light of about 270 nm to about 700 nm, and more preferably about 308 nm to about 320 nm.

The light source may be a simple lamp, or may consist of multiple lamps radiating at different wavelengths. The photoradiation source should be capable of delivering from about 1 j/cm² to at least 120 J/cm^{2.}

In one embodiment, the platelets to be decontaminated to which a pathogen reduction compound and at least one platelet activation inhibitor has been added is flowed past a photoradiation source, and the flow of the material generally provides sufficient turbulence to distribute the pathogen reduction compound and platelet activation inhibitor throughout the fluid to be pathogen reduced. A separate mixing step may optionally be added.

In another embodiment, the fluid, pathogen reduction compound and platelet activation inhibitor/s are placed in a photopermeable container and irradiated in batch mode, preferably while agitating the container to fully distribute the pathogen reduction compound throughout the fluid and expose all the fluid to the radiation. Platelet activation inhibitors may be added to the fluid containing at least platelets and pathogen reduction compound either before the pathogen reduction procedure as describe above, or after the procedure. In this embodiment, the photopermeable container is preferably a blood bag made of transparent or semitransparent plastic, and the agitating means is preferably a mechanism for shaking the container in multiple planes.

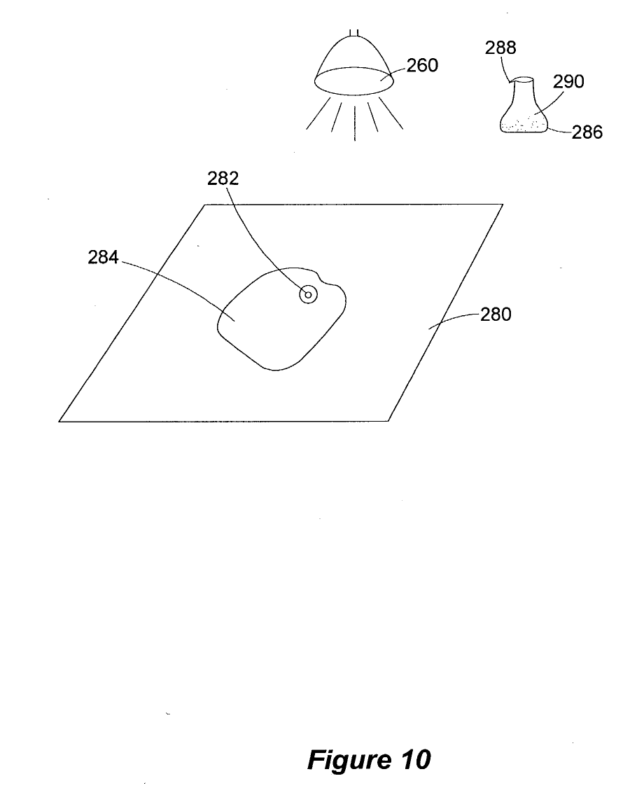

FIG. 10 depicts an embodiment of this invention in which a fluid containing platelets to be decontaminated is placed in a bag 284 equipped with an inlet port 282, through which photosensitizer and platelet activation inhibitors 290 may be added from flask 286 via pour spout 288. Shaker table 280 is activated to agitate the bag 284 to mix the fluid to be decontaminated, the photosensitizer, and the platelet activation inhibitors together while photoradiation source 260 is activated to irradiate the fluid and photosensitizer in bag 284. The photosensitizer and/or platelet activation inhibitors may be added to the container in powdered or liquid form, or alternatively, the bag 284 can be provided prepackaged to contain photosensitizer and/or platelet activation inhibitors and the platelets may thereafter be added to the bag. The platelet activation inhibitors may also be added to bag 284 through a sterile barrier filter (not shown) connected to inlet port 282.

The amount of photosensitizer to be mixed with the platelets will be an amount sufficient to adequately inactivate the reproductive ability of a pathogen. Preferably the photosensitizer is used in a concentration of at least about 1 μM up to the solubility of the photosensitizer in the fluid. For 7,8-dimethyl-10-ribityl isoalloxazine a concentration range between about 1 μM and about 160 μM is preferred, preferably about 50 μM. The photosensitizer may be added directly to the platelets, or may be added in a pre-mixed aqueous solution, for example water, storage buffer or a suspension solution.

Additives additional to platelet inactivation inhibitor additives such as the glycolytic inhibitor 2-deoxy-D-glucose may also be used with the platelet pathogen reduction/storage solution of this invention. In platelets, 2-deoxy-D-glucose slows down the rate of glycolysis by competing with glucose for enzymes utilized in the glycolysis pathway. 2-deoxy-D-glucose is phosphorylated by the same enzymes which phosphorylate glucose, but at a slower rate than that of glucose phosphorylation. Such competitive binding slows the rate of glucose breakdown by the cell and consequently slows the rate of lactic acid production by platelets during storage. Such additives may help contribute to platelet viability during and after a pathogen reduction procedure. 2-deoxy-D-glucose may be added to the pathogen reduction/storage solution in a concentration of about 10 mM.

Quenchers may also be added to the fluid to make the process more efficient and selective. Such quenchers include antioxidants or other agents to prevent damage to desired fluid components or to improve the rate of reduction of pathogens and are exemplified by adenine, histidine, cysteine, tyrosine, tryptophan, ascorbate, N-acetyl-L-cysteine, propyl gallate, glutathione, mercaptopropionylglycine, dithiothreotol, nicotinamide, BHT, BHA, lysine, serine, methionine, glucose, mannitol, vitamin E, trolox, alpha-tocopheral acetate and various derivatives, glycerol, and mixtures thereof. Quenchers may be added to the pathogen reduction/storage solution in an amount necessary to prevent damage to the platelets.

Pathogen reduction methods as described above may be designed as stand-alone units, or may be incorporated into existing apparatuses known to the art for reducing pathogens in blood or blood components. The process is further described in U.S. Pat. Nos. 6,277,337 and 6,258,577 issued to Goodrich et al., which are incorporated by reference herein in their entirety to the amount not inconsistent.

### EXAMPLES

### Example 1

Example 1 shows the results of commonly used measures of platelet cell quality which may be used to measure the effects of platelet activation inhibitors on platelets during pathogen reduction and/or subsequent storage. These may be indirect measures of the increase in intracellular cAMP in pathogen reduced and stored platelets.

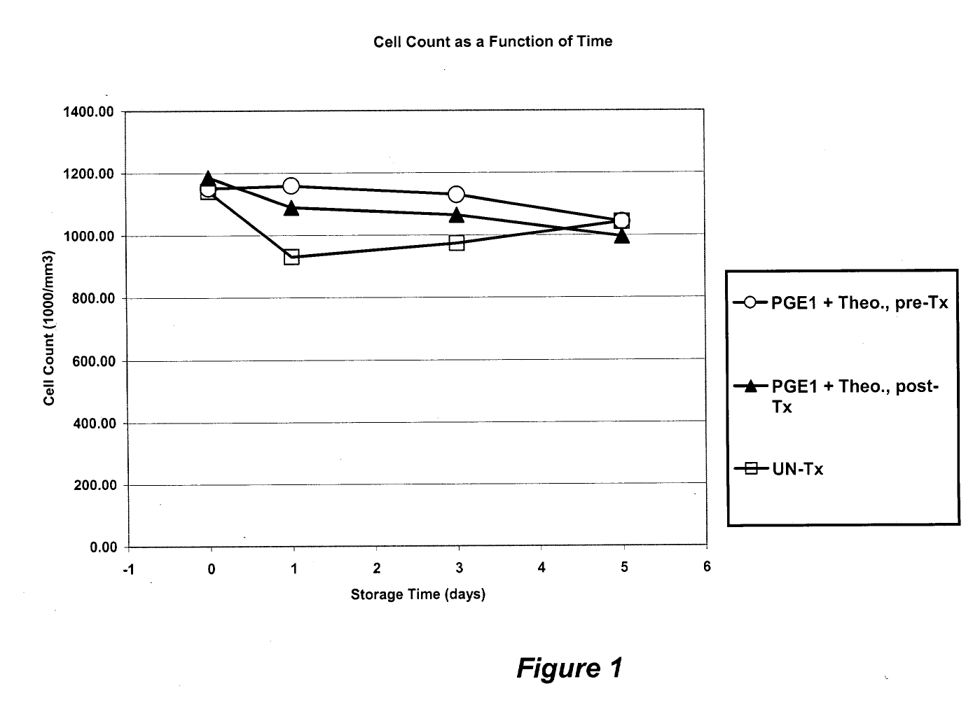

FIGS. 1-5 are graphs showing standard measures of platelet cell quality during 5 days of storage. Platelets were separated from whole blood and collected using a blood collection device such as the COBE Spectra™ or TRIMA® apheresis systems (available from Gambro BCT, Inc., Lakewood, Colo., USA). However, it should be noted that any device known in the art for separating blood into components may be used to collect platelets without departing from the spirit ad scope of the present invention.

The platelet activation inhibitors prostaglandin E1 (PGE₁) and theophylline (Theo) were added either alone or in combination to a solution containing a final concentration of 50 μM riboflavin and saline to make a solution which could be used as a platelet pathogen reduction solution. The resulting pathogen reduction solution was then added to the platelets and incubated for 60 minutes. The pathogen reduction solution and platelets were then irradiated at 12 J/cm². The irradiated platelets were stored for 5 days under standard platelet storage conditions at 22° C., and platelet cell quality was determined.

Untreated, (or control) platelets (designated Un-Tx) were incubated with riboflavin and irradiated, but platelet activation inhibitors were not added. Platelet activation inhibitors were added to platelets either before irradiation (designated pre-Tx) or after irradiation (designated post-Tx). The addition of the platelet activation inhibitors prior to irradiation is designated in the graph legend as PGE₁+Theo., pre-Tx. The addition of the platelet activation inhibitors subsequent to irradiation is designated in the graph legend as PGE₁+Theo., post-Tx. Theophylline was added at a final concentration of 0.95 mM, and PGE₁ was added at a final concentration of 150 mM.

Treated platelets are defined as irradiated platelets to which platelet activation inhibitors were added. The term “treated platelets” encompasses platelets which were irradiated in the presence of a photosensitizer first followed by the addition of activation inhibitors, as well as platelets to which activation inhibitors were first added followed by irradiation.

Platelet cell count, pH, glucose consumption, lactate production, and hypotonic stress response (HSR) were measured to determine indirectly the effect platelet activation inhibitors had on cell quality after the pathogen reduction process and after storage.

FIG. 1 is a graph of the cell count of pathogen reduced platelets over 5 days of storage.

The addition of activation inhibitors to platelets before irradiation substantially prevents platelets from aggregating together following treatment. If platelets become activated and aggregate together, the number of cells which can be counted in the fluid will decrease. Although the starting numbers for both treated and untreated cells are the same at day 0, by day 1, the platelets in solution which did not contain additional platelet activation inhibitor additives show decreased cell numbers, most likely due to platelet clumping (and consequently platelet activation). The addition of platelet activation inhibitors to platelets before exposure to irradiation appears to prevent platelet clumping to a greater extent than adding platelet activation inhibitors post-illumination.

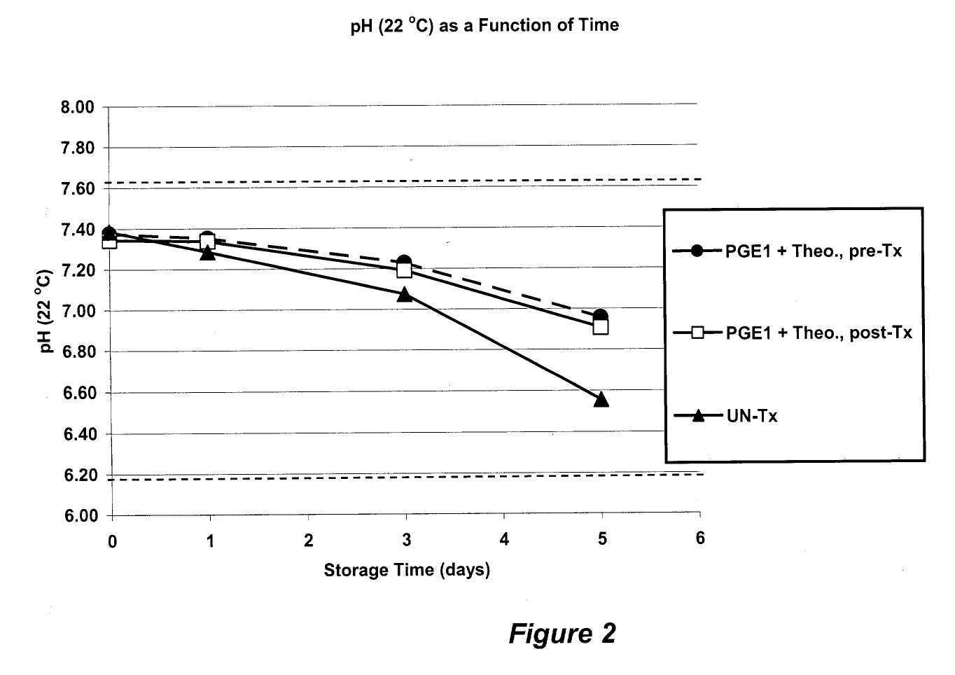

FIG. 2 is a graph showing changes in pH of pathogen reduced platelets over 5 days of storage.

The addition of platelet activation inhibitors both before and after illumination appears to help maintain the pH of the stored platelets as compared to untreated platelets. Maintenance of pH by platelets over time is an indirect measure of platelet cell quality. If the pH of the storage solution drops, platelets have a higher likelihood of becoming activated during the storage process.

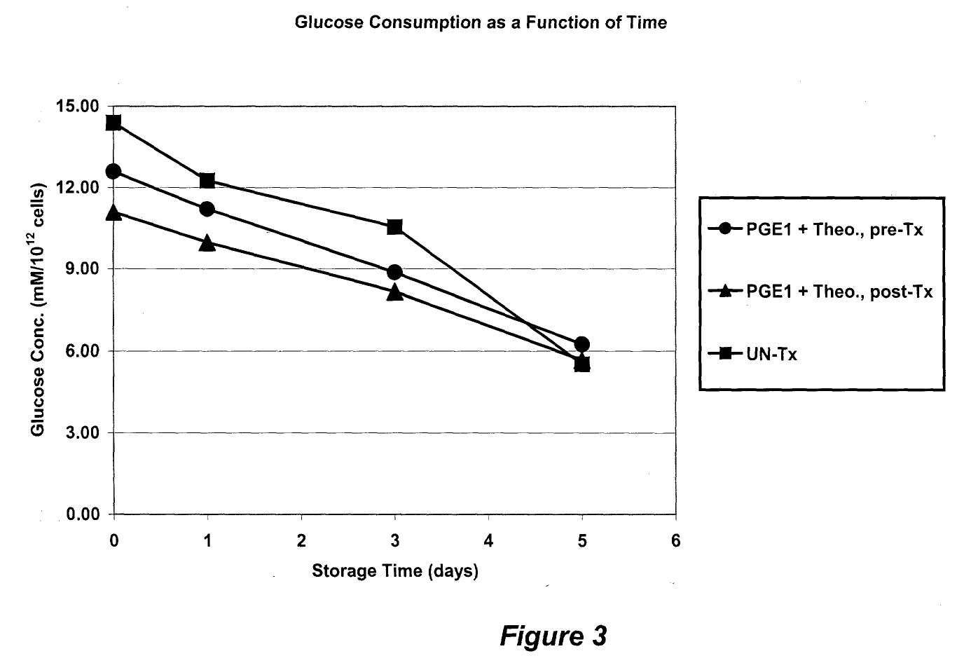

FIG. 3 shows glucose consumption of pathogen reduced platelets over a 5 day storage period.

Glucose is broken down by platelets to produce cellular energy via the glycolytic pathway. To become activated, platelets need to consume greater amounts of energy than in a non-activated state. Therefore, activated platelets consume glucose at a higher rate than non-activated platelets. With the addition of platelet activation inhibitors, platelets remain in a non-activated state, and therefore glucose is consumed at a slower rate by platelets treated with platelet activation inhibitor additives as compared to untreated platelets.

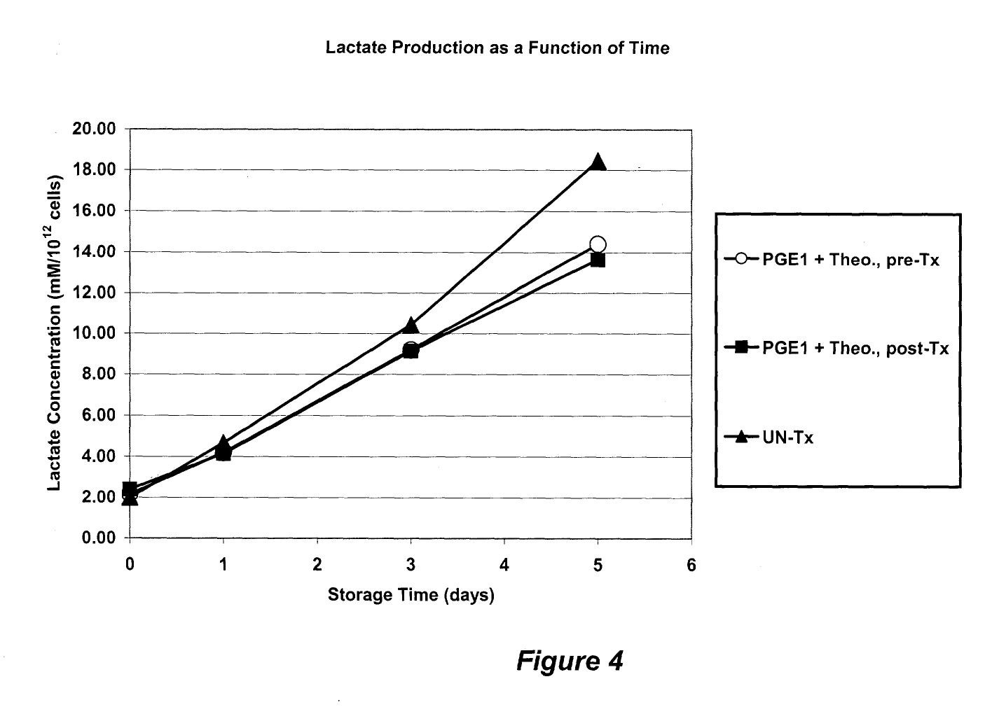

FIG. 4 shows lactate production by platelets during storage.

For every molecule of glucose consumed by a metabolizing platelet, two molecules of lactic acid are produced. If platelets are activated, they consume more glucose and consequently produce more lactic acid. Lactic acid buildup within cells causes the pH of the solution to drop. Such a drop in pH may cause decreased cell quality during storage, and may further platelet activation. As shown in FIG. 4, with the addition of platelet activation inhibitor additives lactic acid (or lactate) is produced by platelets at a slower rate compared to untreated platelets.

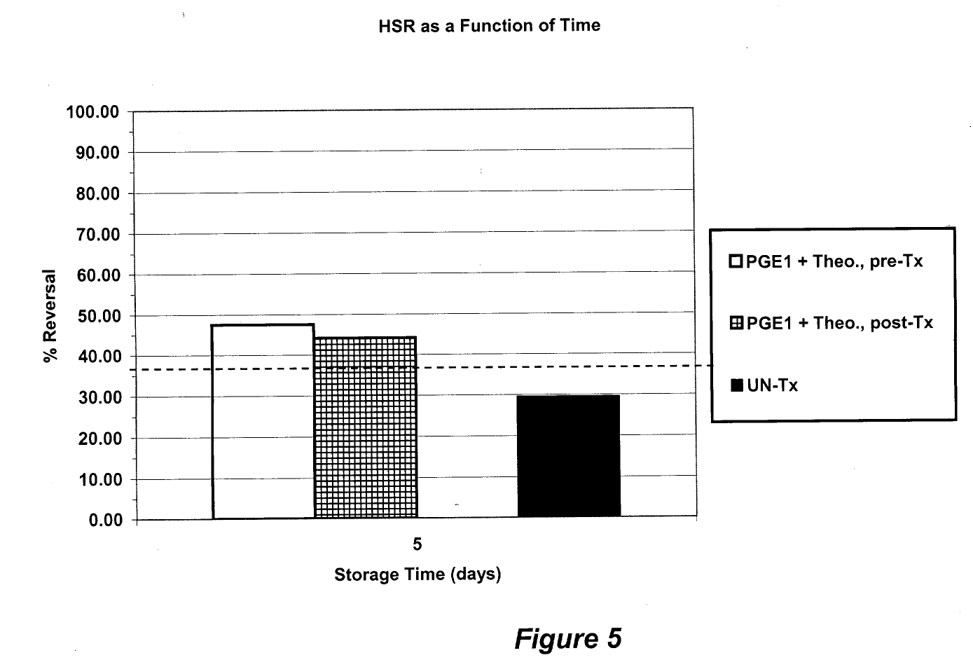

FIG. 5 is a measure of the % reversal from exposure to a hypotonic stress (HSR) of pathogen reduced platelets during 5 days of storage.

As described above, the ability of platelets to overcome the addition of a hypotonic solution is one measure of platelet cell quality after storage. The addition of activation inhibitor additives to platelets pre and post irradiation appears to significantly contribute to the treated platelets ability to show a reversable hypotonic stress response after 5 days of storage compared to untreated platelets.

### Example 2

Other markers of platelet activation after pathogen reduction and storage were also measured, such as cytokine release and cell surface markers of platelet activation. Such markers are another way of measuring the suitability for transfusion purposes of pathogen reduced platelets.

Platelet Surface Antigens

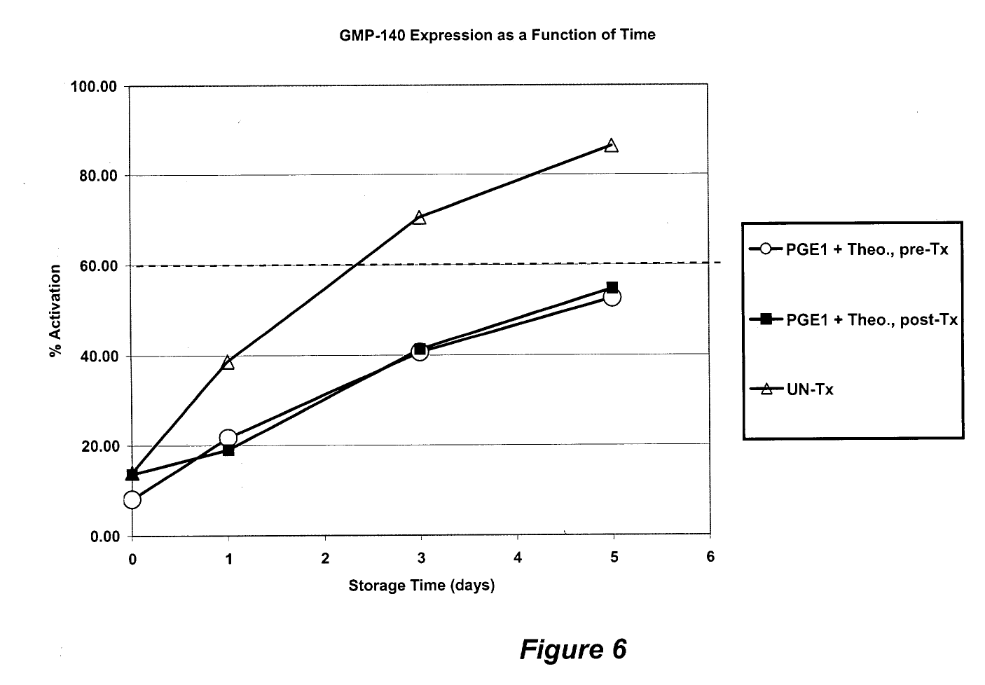

FIG. 6 shows the expression of GMP-140 on the surface of platelets stored for 5 days. GMP-140 is a measure of p-selectin which is a platelet surface protein upregulated upon platelet aggregation. Treatment of platelets with activation inhibitor additives PGE₁ and theophylline in combination before and after a pathogen reduction procedure helps to slow the rate of platelet activation during storage as compared to untreated cells.

Cytokine Measurements

The supernatent of treated and untreated platelets were assayed to determine levels of RANTES (regulated upon activation, normal T-cell expressed and secreted) and VEGF (human vascular endothelial growth factor) produced by treated and untreated platelets. Both cytokines were quantified by a commercially available ELISA (available from R & D Systems, Wiesbaden-Nordenstadt, Germany) according to manufacturer's instructions.

The chemokine RANTES is known to be released from activated platelets. VEGF has also been reported to be released upon platelet activation. The presence of enhanced levels of VEGF and RANTES could be additional markers used to determine the transfusion efficacy of platelets which have undergone a pathogen reduction procedure in the presence of platelet activation inhibitors.

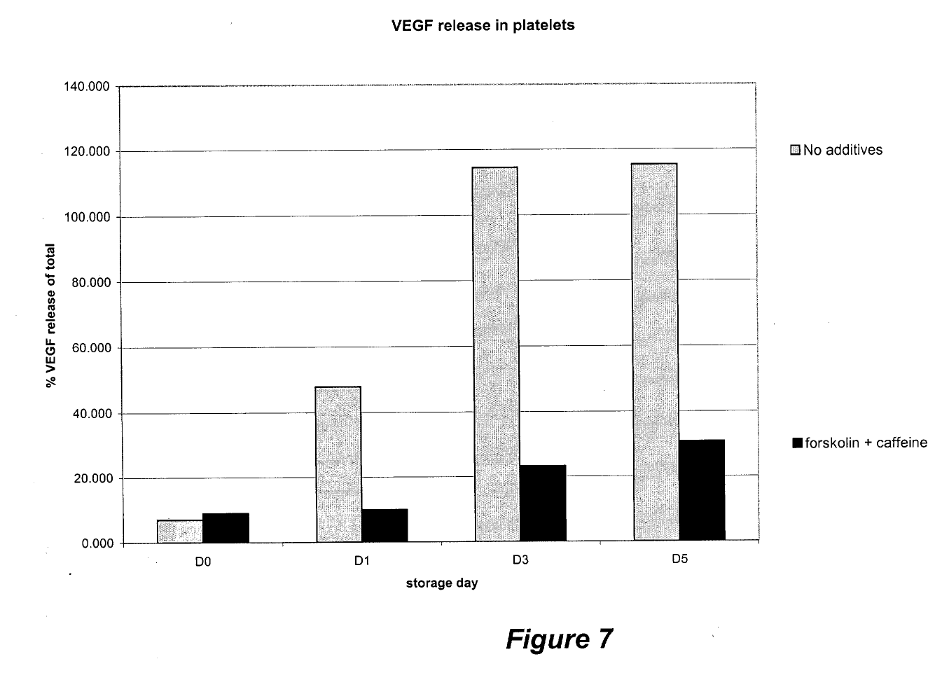

FIG. 7 measures VEGF released by pathogen reduced platelets over five days of storage in the presence or absence of platelet activator inhibitor additives. Platelets were irradiated with 12 J/cm² light in the presence of riboflavin either with or without the platelet activation inhibitor additives forskolin and caffeine. 3 μM forskolin and 1.9 mM caffeine were added to the platelets. As shown, the presence of forskolin and caffeine substantially reduced the release of VEGF by platelets over five days of storage.

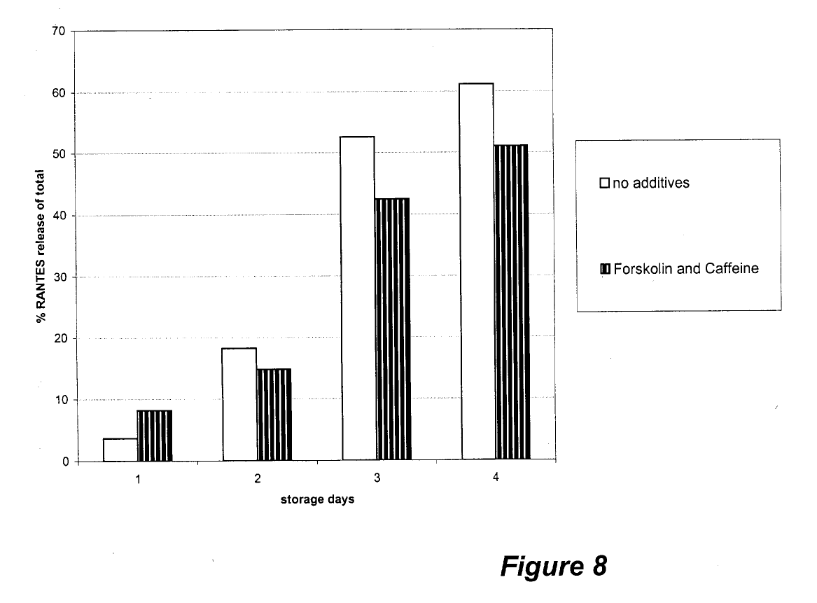

FIG. 8 is a measure of RANTES released by pathogen reduced and stored platelets. FIG. 8 compares the amount of RANTES released by platelets which have been irradiated at 12 J/cm² in the presence of riboflavin, which were than stored over five days in a solution which contined no additional platelet activation inhibitor additives as compared to platelets stored in a solution containing 0.95 mM caffeine and 3 mM forskolin. As shown, the combination of an adenylate cyclase stimulator (forskolin) and a phosphodiesterase inhibitor (caffeine) to a platelet storage solution results in a decrease in the amount of RANTES released by platelets as compared to platelets stored in a solution which does not contain additional platelet activation inhibitor additives.

cAMP

The effects of platelet activation inhibitors on the upregulation of cAMP by platelets which were pathogen reduced and stored can be measured directly using commercially available cAMP detection kits. One such kit which may be used is a fluorescent assay for cAMP available from Cayman Chemical (Ann Arbor, Mich., USA).

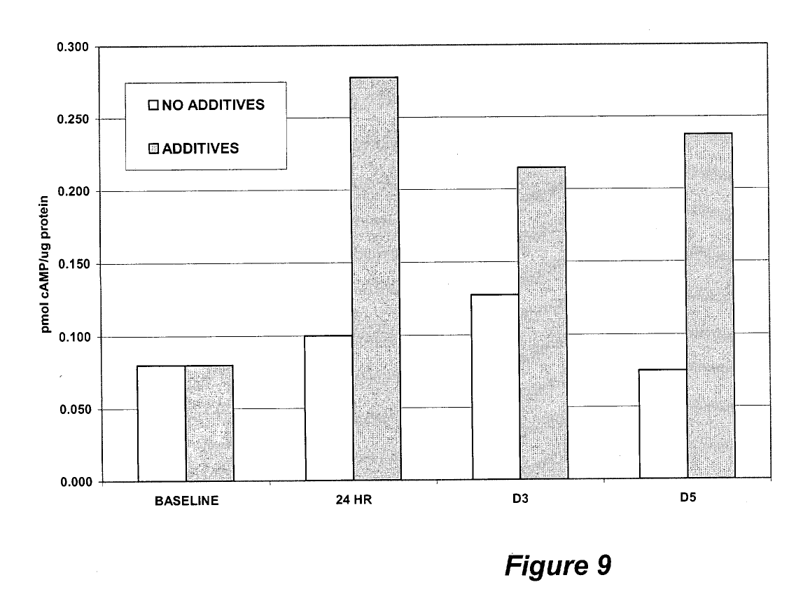

FIG. 9 is a direct measurement of cAMP produced by platelets over five days of storage. cAMP was measured according to manufacturer's instructions. Platelets which were pathogen reduced and stored in solutions containing 30 μM forskolin and 1.0 mM caffeine continued to maintain sufficient cAMP levels, thus presumably not becoming activated during storage.

In describing the chemicals which have shown utility as platelet activation inhibitors, it must be understood that the actual chemicals mentioned together with functionally equivalent materials are intended to be within the scope of this invention. Chemicals that are known to applicants to have known or demonstrated utility as platelet activation inhibitors have been specifically set forth in the instant application. However, it is intended that the scope of the application be extended to other functionally effective chemicals, both existing chemicals and chemicals yet to be discovered.

Platelet activation inhibitors containing adenylate cyclase stimulators and phosphodiesterase inhibitors may be added either alone or in combination to a solution containing platelets to be pathogen reduced. PGE₁, forskolin, theophylline and caffeine either alone or in combination may be added.

It is also understood that other platelet additives such as 2-deoxy-D-glucose may also be added to improve platelet storage conditions. Quenchers or a combination of quenchers as set forth above may also be used.

In one embodiment, the present invention contemplates an aqueous synthetic pathogen reduction solution comprising saline, a photosensitizer and at least one platelet activation inhibitor. Water or a physiological buffer may replace saline in the pathogen reduction solution.

### Example 3

In an alternative embodiment, the platelet activation inhibitor additives may be added to any commercially available synthetic platelet storage solutions after the pathogen reduction process, to aid in long term platelet storage. The solutions listed below contain the photosensitizer isoalloxazine, but any pathogen reduction compound may be used.

This example compares novel blood component additive solutions for addition to platelets separated from whole blood. Six commercially available solutions were used: PAS II, PSMI-pH, PlasmaLyte A, SetoSol, PAS III, and PAS. To each known solution was added an effective amount of an endogenous photosensitizer, 7,8-dimethyl-10-ribityl isoalloxazine as well as an effective amount of at least one platelet activation inhibitor such as PGE₁ and/or theophylline or other methylxanthine. For 7,8-dimethyl-10-ribityl isoalloxazine a final or working concentration range between about 1 μM and about 160 μM is preferred, preferably about 50 μM. The composition of each solution is shown in Table 3a below, and varies in the amount of blood component additives present. The blood additive components may be in a physiological solution, as well as a dry medium adapted to be mixed with a solvent, including tablet, pill or capsule form.

TABLE 3a

| Blood | | | | | | |
|---|---|---|---|---|---|---|
| Component | Platelet Storage Solution | | | | | |
| Additive | PSS 1 | PSS 2 | PSS 3 | PSS 4 | PSS 5 | PSS 6 |
| KCl (mM) | | 5.0 | 5.0 | 5.0 | 5.1 | |
| CaCl₂ (mM) | | | | | 1.7 | |
| MgCl₂ (mM) | | | 3.0 | 3.0 | | |
| MgSO₄ (mM) | | | | | 0.8 | |
| sodium | 10.0 | 23.0 | 23.0 | 17.0 | 15.2 | 12.3 |
| citrate (mM) | | | | | | |
| citric | | | | | 2.7 | |
| acid (mM) | | | | | | |
| NaHCO₃ (mM) | | | | | 35.0 | |
| Na₂HPO₄ | | 25.0 | | 25.0 | 2.1 | 28.0 |
| (mM) | | | | | | |
| sodium | 30.0 | | 27.0 | 23.0 | | 42.0 |
| acetate (mM) | | | | | | |
| sodium | | | 23.0 | | | |
| gluconate | | | | | | |
| (mM) | | | | | | |
| glucose (mM) | | | | 23.5 | 38.5 | |
| maltose (mM) | | | | 28.8 | | |
| 7,8-dimethyl | 50.0 | 50.0 | 50.0 | 50.0 | 50.0 | 50.0 |
| 10-ribityl | | | | | | |
| isoalloxazine | | | | | | |
| (μM) | | | | | | |
| platelet | variable | variable | variable | variable | variable | variable |
| activation | | | | | | |
| inhibitor | | | | | | |

In Example 3, the platelet storage solution PSS 1 (also known as PAS II) comprises a physiological saline solution, tri-sodium citrate at a concentration of approximately about 10 mM, sodium acetate at a concentration of approximately about 30 mM, 7, 8-dimethyl-10-ribityl isoalloxizine at a concentration of about 50 μM and an effective amount of at least one platelet activation inhibitor.

The platelet storage solution PSS 2 (also known as PSMI-pH) comprises a physiological saline solution, potassium chloride at a concentration of approximately about 5 mM, tri-sodium citrate at a concentration of approximately about 23 mM, a mixture of monosodium phosphate and dibasic sodium phosphate at a concentration of approximately about 25 mM, 7, 8-dimethyl-10-ribityl isoalloxizine at a concentration of about 50 μM and an effective amount of at least one platelet activation inhibitor.

The platelet storage solution PSS 3 (also known as PlasmaLyte A) comprises a physiological saline solution, potassium chloride at a concentration of approximately about 5 mM, magnesium chloride at a concentration of approximately about 3 mM, tri-sodium citrate at a concentration of approximately about 23 mM, sodium acetate at a concentration of approximately about 27 mM, sodium gluconate at a concentration of approximately about 23 mM, 7, 8-dimethyl-10-ribityl isoalloxizine at a concentration of about 50 μM and an effective amount of at least one platelet activation inhibitor.

Platelet storage solution PSS 4 (also known as SetoSol) comprises a physiological saline solution, potassium chloride at a concentration of approximately about 5 mM, magnesium chloride at a concentration of approximately about 3 mM, tri-sodium citrate at a concentration of approximately about 17 mM, sodium phosphate at a concentration of approximately about 25 mM, sodium acetate at a concentration of approximately about 23 mM, glucose at a concentration of approximately about 23.5 mM, maltose at a concentration of approximately about 28.8 mM, 7,8-dimethyl-10-ribityl isoalloxizine at a concentration of about 50 μM and an effective amount of at least one platelet activation inhibitor.

Platelet storage solution PSS 5 (also known as PAS III) comprises a physiological saline solution, potassium chloride at a concentration of approximately about 5.1 mM, calcium chloride at a concentration of approximately about 1.7 mM, magnesium sulfate at a concentration of approximately about 0.8 mM, tri-sodium citrate at a concentration of approximately about 15.2 mM, citric acid at a concentration of approximately about 2.7 mM, sodium bicarbonate at a concentration of approximately about 35 mM, sodium phosphate at a concentration of approximately about 2.1 mM, glucose at a concentration of approximately about 38.5 mM, 7,8-dimethyl-10-ribityl isoalloxizine at a concentration of about 50 μM and an effective amount of at least one platelet activation inhibitor.

Platelet storage solution PSS 6 (also known as PAS) comprises a physiological saline solution, tri-sodium citrate at a concentration of approximately about 12.3 mM, sodium phosphate at a concentration of approximately about 28 mM, sodium acetate at a concentration of approximately about 42 mM, 7,8-dimethyl-10-ribityl isoalloxizine at a concentration of about 50 μM and an effective amount of at least one platelet activation inhibitor.

In an aspect of this embodiment, physiologic saline may be replaced with a solvent comprising water and an effective amount of sodium chloride.

The blood additive solution may also comprise any other synthetic additive solution including an effective amount of 7,8-dimethyl-10-ribityl isoalloxazine and a platelet activation inhibitor in a liquid, pill or dry medium form. PSS 7, PSS 8 and PSS 9 (set forth in Table 3b below) are further examples of platelet pathogen reduction and/or storage solutions which may be used in the present invention.

TABLE 3b

| | Platelet Storage Solution | | |
|---|---|---|---|
| Blood Component Additive | PSS 7 | PSS 8 | PSS 9 |
| NaCl (mM) | 115.0 | 78.3 | 68.5 |
| potassium cloride (mM) | | 5.7 | 5.0 |
| MgCl₂ (mM) | | 1.7 | 1.5 |
| sodium citrate (mM) | 10.0 | | |
| sodium phosphate (monobasic) | 6.2 | 5.4 | 8.5 |
| sodium phosphate (dibasic) | 19.8 | 24.6 | 21.5 |
| sodium acetate (mM) | 30.0 | 34.3 | 30.0 |
| 7,8-dimethyl 10-ribityl isoalloxazine (μM) | 14.0 | variable | 14.0 |
| platelet activation inhibitor | variable | variable | variable |

As described in Table 3b, PSS 7 was prepared in RODI water and sodium chloride at a concentration of approximately 115 mM, sodium citrate at a concentration of approximately 10.0 mM, sodium phosphate (monobasic) at a concentration of approximately 6.2 mM, sodium phosphate (dibasic) at a concentration of approximately 19.8 mM, sodium acetate at a concentration of approximately 30.0 mM, 7,8-dimethyl 10-ribityl isoalloxazine at a concentration of approximately 14.0 μM and an effective amount of at least one platelet activation inhibitor. PSS 7 has a pH of 7.2.

PSS 8 was prepared in RODI water and comprises and sodium chloride at a concentration of approximately 78.3 mM, potassium chloride at a concentration of approximately 5.7 mM, magnesium chloride at a concentration of approximately 1.7 mM, sodium phosphate (monobasic) at a concentration of approximately 5.4 mM, sodium phosphate (dibasic) at a concentration of approximately 24.6 mM, sodium acetate at a concentration of approximately 34.3 mM, a variable concentration of 7,8-dimethyl 10-ribityl isoalloxazine and an effective amount of at least one platelet activation inhibitor. PSS 8 has a pH of 7.4, and an osmolarity of 297 mmol/kg.

PSS 9 was prepared in RODI water and comprises and sodium chloride at a concentration of approximately 68.5 mM, potassium chloride at a concentration of approximately 5.0 mM, magnesium chloride at a concentration of approximately 1.5 mM, sodium phosphate (monobasic) at a concentration of approximately 8.5 mM, sodium phosphate (dibasic) at a concentration of approximately 21.5 mM, sodium acetate at a concentration of approximately 30.0 mM, 7,8-dimethyl 10-ribityl isoalloxazine at a concentration of approximately 14.0 μM and an effective amount of at least one platelet activation inhibitor. PSS 9 has a pH of 7.2, and an osmolarity of 305 mmol/kg.

While the compositions and methods of this invention have been described in terms of preferred embodiments, it will be apparent to those of skill in the art that variations may be applied to the processes described herein without departing from the concept, spirit and scope of the invention. All such similar substitutions and modifications apparent to those skilled in the art are deemed to be within the spirit, scope and concept of the invention as it is set out in the following claims.

1. A method for maintaining the suitability for transfusion purposes of platelets which may contain pathogens during a pathogen reduction process comprising the steps of:
adding a pathogen reducing compound to the platelets;
adding to the platelets and pathogen reducing compound at least one platelet activation inhibitor additive; and
exposing the platelets and pathogen reducing compound and at least one platelet activation inhibitor additive to light of a sufficient wavelength to reduce any pathogens contained in the platelets.

2. The method of claim 1 wherein the step of adding to the platelets and pathogen reducing compound at least one platelet activation inhibitor additive occurs prior to the exposing step.

3. The method of claim 1 wherein the step of adding to the platelets and pathogen reducing compound at least one platelet activation inhibitor additive occurs subsequent to the exposing step.

4. The method of claim 1 wherein the at least one platelet activation inhibitor additive is an adenylate cyclase stimulator in an amount effective to increase the production of adenosine 3′, 5′ cyclic phosphate in the platelets.

5. The method of claim 1 wherein the adenylate cyclase stimulator is selected from the group consisting of PGE₁ and forskolin.

6. The method of claim 5 wherein PGE₁ is added at a final concentration of between about 100 nM and 500 nM.

7. The method of claim 6 wherein PGE₁ is added at a final concentration of 150 nM.

8. The method of claim 5 wherein forskolin is added at a final concentration of between about 1 μM and 100 μM.

9. The method of claim 8 wherein forskolin is added at a final concentration of about 3 μM.

10. The method of claim 1 wherein the at least one platelet activation inhibitor additive is a phosphodiesterase inhibitor in an amount effective to reduce the degradation of cAMP in the platelets.

11. The method of claim 10 wherein the phosphodiesterase inhibitor is a methylxanthine.

12. The method of claim 10 wherein the phosphodiesterase inhibitor is selected from the group consisting of xanthine, theophylline, caffeine, theobromine, aminophylline, oxtriphylline, dyphylline, pentoxifylline, isobutulmethylxanthine, dipyramole, and papaverine.

13. The method of claim 11 wherein the methylxanthine is theophylline added at a final concentration of between about 0.90 mM and 2.2 mM.

14. The method of claim 13 wherein the theophylline is added at a final concentration of about 0.95 mM.

15. The method of claim 11 wherein the methylxanthine is caffeine added at a final concentration of between about 0.9 mM and 2.2 mM.

16. The method of claim 15 wherein the caffeine is added at a final concentration of about 0.95 mM.

17. The method of claim 1 wherein the step of adding to the platelets and pathogen reducing compound at least one platelet activation inhibitor additive further comprises adding a second platelet activation inhibitor additive.

18. The method of claim 17 wherein the at least one platelet activation inhibitor additive comprises an adenylate cyclase stimulator and the second platelet activation inhibitor additive comprises a phosphodiesterase inhibitor.

19. The method of claim 1 wherein the step of adding to the platelets and pathogen reducing compound at least one platelet activation inhibitor additive further comprises adding a glycolytic inhibitor.

20. The method of claim 19 wherein the glycolytic inhibitor is 2-deoxy-D-glucose.

21. The method of claim 1 wherein the step of adding to the platelets and pathogen reducing compound at least one platelet activation inhibitor additive further comprises adding at least one quencher.

22. The method of claim 21 wherein the quencher is selected from the group consisting of adenine, histidine, cysteine, tyrosine, tryptophan, ascorbate, N-acetyl-L-cysteine, propyl gallate, glutathione, mercaptopropionylglycine, dithiothreotol, nicotinamide, BHT, BHA, lysine, serine, methionine, glucose, mannitol, vitamin E, trolox, alpha-tocopheral acetate and various derivatives, glycerol, and mixtures thereof.

23. The method of claim 1 wherein the pathogen reducing compound comprises a photosensitizer.

24. The method of claim 23 wherein the photosensitizer is an endogenous photosensitizer.

25. The method of claim 24 wherein the endogenous photosensitizer is riboflavin.

26. A storage solution for maintaining the cell quality of pathogen reduced platelets during storage comprising:
a pathogen reducing compound; and
at least one platelet activation inhibitor additive.

27. The storage solution of claim 26 wherein the storage solution may also be used as a pathogen reduction solution.

28. The storage solution of claim 26 wherein the at least one platelet activation inhibitor comprises an adenylate cyclase stimulator in an amount effective to increase the production of adenosine 3′, 5′ cyclic phosphate in the platelets.

29. The storage solution of claim 28 wherein the adenylate cyclase stimulator is selected from the group consisting of PGE₁ and forskolin.

30. The storage solution of claim 29 wherein PGE₁ is added at a final concentration of between about 100 nM and 500 nM.

31. The storage solution of claim 30 wherein PGE₁ is added at a final concentration of 150 nM.

32. The storage solution of claim 29 wherein forskolin is added at a final concentration of between about 1 μM and 100 μM.

33. The storage solution of claim 32 wherein forskolin is added at a final concentration of about 3 μM.

34. The storage solution of claim 26 wherein the at least one platelet activation inhibitor additive is a phosphodiesterase inhibitor in an amount effective to reduce the degradation of cAMP in the platelets.

35. The storage solution of claim 34 wherein the phosphodiesterase inhibitor is a methylxanthine.

36. The storage solution of claim 34 wherein the phosphodiesterase inhibitor is selected from the group consisting of xanthine, theophylline, caffeine, theobromine, aminophylline, oxtriphylline, dyphylline, pentoxifylline, isobutulmethylxanthine, dipyramole, and papaverine.

37. The storage solution of claim 35 wherein the methylxanthine is theophylline added at a final concentration of between about 0.90 mM and 2.2 mM.

38. The storage solution of claim 37 wherein the theophylline is added at a final concentration of about 0.95 mM.

39. The storage solution of claim 35 wherein the methylxanthine is caffeine added at a final concentration of between about 0.90 mM and 2.2 mM.

40. The storage solution of claim 39 wherein the caffeine is added at a final concentration of about 0.95 mM.

41. The storage solution of claim 26 wherein the at least one platelet activation inhibitor additive further comprises a second platelet activation inhibitor additive.

42. The storage solution of claim 41 wherein the at least one platelet activation inhibitor additive comprises an adenylate cyclase stimulator and the second platelet activation inhibitor additive comprises a phosphodiesterase inhibitor.

43. The storage solution of claim 26 further comprising a glycolytic inhibitor.

44. The storage solution of claim 43 wherein the glycolytic inhibitor is 2-deoxy-D-glucose.

45. The storage solution of claim 26 further comprising at least one quencher.

46. The storage solution of claim 45 wherein the quencher is selected from the group consisting of adenine, histidine, cysteine, tyrosine, tryptophan, ascorbate, N-acetyl-L-cysteine, propyl gallate, glutathione, mercaptopropionylglycine, dithiothreotol, nicotinamide, BHT, BHA, lysine, serine, methionine, glucose, mannitol, vitamin E, trolox, alpha-tocopheral acetate and various derivatives, glycerol, and mixtures thereof.

47. The storage solution of claim 26 wherein the pathogen reducing compound comprises a photosensitizer.

48. The storage solution of claim 47 wherein the photosensitizer is an endogenous photosensitizer.

49. The storage solution of claim 48 wherein the endogenous photosensitizer is riboflavin.

50. The storage solution of claim 26 further comprising a solvent.

51. The storage solution of claim 50 wherein the solvent is selected from the group consisting of PSS1, PSS2, PSS3, PSS4, PSS5, PSS6, PSS7, PSS8 and PSS9.

52. The storage solution of claim 50 wherein the solvent is selected from the group consisting of saline and water.

53. A fluid for transfusing into a recipient comprising:
platelets;
a pathogen reduction compound; and
one or more platelet activation inhibitor additives.
